(19) United States
(12) Patent Application Publication
(10) Pub. No.: US 2023/0041402 A1
(43) Pub. Date: Feb. 9, 2023

(54) ULTRASOUND IMAGING DEVICE, METHOD OF OPERATING ULTRASOUND IMAGING DEVICE, COMPUTER-READABLE RECORDING MEDIUM, AND ULTRASOUND IMAGING SYSTEM

(71) Applicant: OLYMPUS CORPORATION, Tokyo (JP)

(72) Inventor: Hidenori TSUBOI, Yokohama-shi (JP)

(73) Assignee: OLYMPUS CORPORATION, Tokyo (JP)

(21) Appl. No.: 17/971,837

(22) Filed: Oct. 24, 2022

Related U.S. Application Data
(63) Continuation of application No. PCT/JP2020/025680, filed on Jun. 30, 2020.

(51) Int. Cl. A61B 8/12; B06B 1/02; B06B 1/06

(52) U.S. Cl. CPC A61B 8/12; B06B 1/0215; B06B 1/0607; B06B 2201/76

(57) ABSTRACT

An ultrasound imaging device includes: a first transmitter configured to transmit a transmission signal to at least one piezoelectric element; a receiver configured to receive a reception signal from the at least one piezoelectric element; a second transmitter configured to transmit a given signal to the at least one piezoelectric element; a timing controller configured to control a transmitting timing at which the first transmitter transmits the transmission signal and a receiving timing at which the receiver receives the reception signal; and a signal controller configured to cause the second transmitter to transmit the given signal to a first area to which the first transmitter does not transmit the transmission signal at the transmitting timing or cause the second transmitter to transmit the given signal to a second area from which the receiver does not receive the reception signal at the receiving timing.

### CROSS-REFERENCE TO RELATED APPLICATION

This application is a continuation of International Application No. PCT/JP2020/025680, filed on Jun. 30, 2020, the entire contents of which are incorporated herein by reference.

### BACKGROUND

### 1. Technical Field

The present disclosure relates to an ultrasound imaging device, a method of operating an ultrasound imaging device, a computer-readable recording medium, and an ultrasound imaging system.

### 2. Related Art

Ultrasound imaging devices that transmit a transmission signal to an ultrasound transducer to apply ultrasound to a subject, that receive a reception signal that is received by the ultrasound transducer, and that generate an ultrasound image have been known.

An ultrasound imaging device transmits and receives ultrasound using polarization characteristics of piezoelectric elements that an ultrasound transducer includes. Specifically, the ultrasound imaging device applies a transmission signal that is a high-voltage pulse signal to the piezoelectric elements, thereby causing application of ultrasound to a subject from the piezoelectric elements. Thereafter, the piezoelectric elements receive ultrasound echoes that are reflected by the subject and the ultrasound imaging device receives a reception signal that is obtained by converting the ultrasound echoes into a voltage and outputting the voltage. The ultrasound imaging device generates an ultrasound image using the received reception signal.

The polarization characteristics of the piezoelectric elements deteriorate (depolarization) over time and acoustic characteristics deteriorate simultaneously. Japanese Laid-open Patent Publication No. 2011-5024 and Japanese Laid-open Patent Publication No. 2004-230033 disclose techniques of applying a high voltage to piezoelectric elements that are depolarized and thus repolarizing the piezoelectric elements, thereby recovering the acoustic characteristics.

In the technique according to Japanese Laid-open Patent Publication No. 2011-5024, it is necessary to perform a repolarization process regularly at given intervals (at the time of maintenance or starting the ultrasound imaging device). In the technique according to

Japanese Laid-open Patent Publication No. 2004-230033, a high voltage for repolarizing piezoelectric elements is applied when an ultrasound probe is connected to an ultrasound imaging device.

### SUMMARY

In some embodiments, an ultrasound imaging device includes: a first transmitter configured to transmit a transmission signal to at least one piezoelectric element; a receiver configured to receive a reception signal from the at least one piezoelectric element; a second transmitter configured to transmit a given signal to the at least one piezoelectric element; a timing controller configured to control a transmitting timing at which the first transmitter transmits the transmission signal and a receiving timing at which the receiver receives the reception signal; and a signal controller configured to cause the second transmitter to transmit the given signal to a first area to which the first transmitter does not transmit the transmission signal at the transmitting timing or cause the second transmitter to transmit the given signal to a second area from which the receiver does not receive the reception signal at the receiving timing.

In some embodiments, provided is a method of operating an ultrasound imaging device including a first transmitter configured to transmit a transmission signal to at least one piezoelectric element; a receiver configured to receive a reception signal from the at least one piezoelectric element; and a second transmitter configured to transmit a given signal that repolarizes the piezoelectric element to the at least one piezoelectric element. The method includes: by a timing controller, controlling a transmitting timing at which the first transmitter transmits the transmission signal and a receiving timing at which the receiver receives the reception signal; and by a signal controller, causing the second transmitter to transmit the given signal to a first area to which the first transmitter does not transmit the transmission signal at the transmitting timing or causing the second transmitter to transmit the given signal to a second area from which the receiver does not receive the reception signal at the receiving timing.

In some embodiments, provided is a non-transitory computer-readable recording medium with an executable program stored thereon. The program causes an ultrasound imaging device to execute: causing a first transmitter to transmit a transmission signal to at least one piezoelectric element; causing a receiver to receive a reception signal from the at least one piezoelectric element; causing a second transmitter to transmit a given signal to the at least one piezoelectric element; causing a timing controller to control a transmitting timing at which the first transmitter transmits the transmission signal and a receiving timing at which the receiver receives the reception signal; and causing a signal controller to cause the second transmitter to transmit the given signal to a first area to which the first transmitter does not transmit the transmission signal at the transmitting timing or cause the second transmitter to transmit the given signal to a second area from which the receiver does not receive the reception signal at the receiving timing.

In some embodiments, an ultrasound imaging system includes: an ultrasound probe including at least two ultrasound transducers; and a processor configured to cause the at least two ultrasound transducers included in a first area to transmit and receive an ultrasound at a first timing, and transmit a repolarization signal to the at least two ultrasound transducers included in a second area at the first timing.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating an entire configuration of an ultrasound imaging system including an ultrasound imaging device according to a first embodiment;

FIG. 2 is a block diagram illustrating a configuration of the ultrasound imaging device illustrated in FIG. 1;

FIG. 3 is a flowchart illustrating an overview of a process that is executed by the ultrasound imaging device;

FIG. 4 is a diagram for describing a relationship of connection between piezoelectric elements and transceiver circuits;

FIG. 5 is a block diagram illustrating a configuration of an ultrasound imaging device according to a modification;

FIG. 6 is a timing chart presenting timing at which each signal is transmitted or received;

FIG. 7 is a diagram for describing a relationship of connection of the piezoelectric elements and the transceiver circuits at transmitting timing;

FIG. 8 is a diagram for describing a relationship of connection of the piezoelectric elements and the transceiver circuits at receiving timing; and

FIG. 9 is a diagram for describing a positional relationship of a piezoelectric element.

### DETAILED DESCRIPTION

Embodiments of an ultrasound imaging device, an ultrasound imaging system, and a method of operating an ultrasound imaging device will be described below with reference to the accompanying drawings. Note that these embodiments do not limit the disclosure. The disclosure is applicable to ultrasound imaging devices, ultrasound imaging systems, and methods of operating an ultrasound imaging device generally.

In the illustration of the drawings, the same or corresponding components are denoted with the same reference numeral as appropriate. The drawings are schematic and it is necessary to note that the correlation among components in size and the ratio among the components may differ from actual ones. The drawings may contain components whose correlation in size and whose ratio differ among the drawings.

### First Embodiment

### Configuration of Ultrasound Imaging System

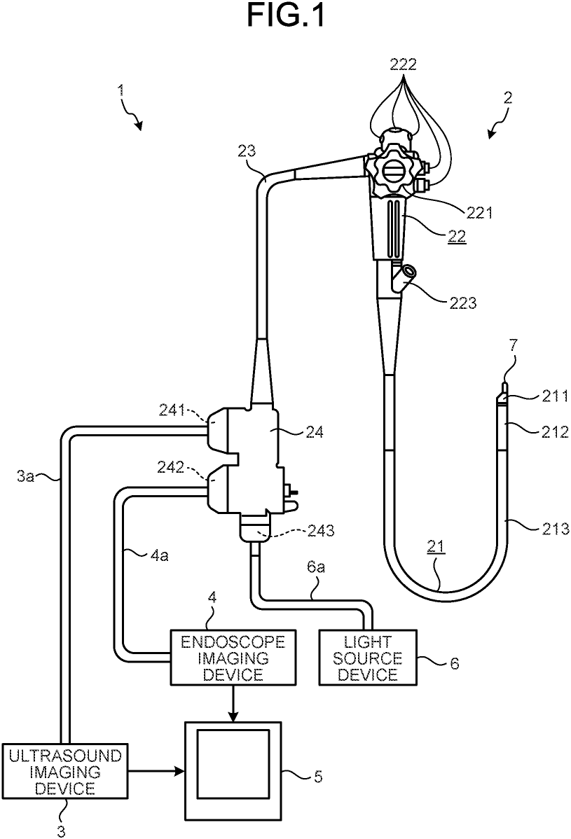

FIG. 1 is a schematic diagram illustrating an entire ultrasound imaging system including an ultrasound imaging device according to a first embodiment. An ultrasound imaging system 1 is a system that performs internal ultrasound observation on a subject, such as a person, using an ultrasound endoscope. As illustrated in FIG. 1, the ultrasound imaging system 1 includes an ultrasound endoscope 2, an ultrasound imaging device 3, an endoscope imaging device 4, a display 5, a light source device 6, and an ultrasound transducer 7.

The ultrasound endoscope 2 includes the ultrasound transducer 7 at its distal end part and converts an electric pulse signal (transmission signal) that is received from the ultrasound imaging device 3 into ultrasound pulses (acoustic pulses) and applies the ultrasound pulses to the subject and converts the ultrasound echoes that are reflected by the subject into an electric echo signal (reception signal) expressing the ultrasound echoes by changes in voltage and outputs the echo signal.

The ultrasound endoscope 2 generally includes an imaging optical system and an imaging element and is inserted into a digestive tract (esophagus, stomach, duodenum or large intestine) or a respiratory organ (trachea or bronchi) of the subject and thus is capable of capturing images of the digestive tract or the respiratory organ. It is also possible to capture images of organs around the digestive tract or the respiratory organ (such as pancreas, gallbladder, bile duct, the biliary tract, lymph nodes, the organ in the mediastinum, blood vessels) using ultrasound. The ultrasound endoscope 2 includes a light guide that guides illumination light that is applied to the subject when optical imaging is performed. While a distal end part of the light guide reaches a distal end of an insertion unit of the ultrasound endoscope 2 to be inserted into the subject, a proximal end part of the light guide is connected to the light source device 6 that generates illumination light.

As illustrated in FIG. 1, the ultrasound endoscope 2 includes an insertion unit 21, an operation unit 22, a universal cord 23, and a connector 24. The insertion unit 21 is a part that is inserted into the subject. As illustrated in FIG. 1, the insertion unit 21 includes a distal end rigid member 211 that is provided on a distal end side and that holds the ultrasound transducer 7 that transmits and receives ultrasound, a curve part 212 that is joined to a proximal end side of the distal end rigid member 211 and that is able to curve, and a flexible tubular part 213 that is joined to a proximal end side of the curve part 212 and that has flexibility. Although specific illustration in the drawing is omitted, in the insertion unit 21, the light guide that transmits the illumination light that is supplied from the light source device 6 and a plurality of signal cables that transmit various signals are arranged and a treatment tool insertion path for inserting a treatment tool is formed. The side of the insertion unit 21 with respect to the ultrasound transducer 7 is referred to as the distal end side and the side on which the insertion unit 21 is continuous to the operation unit 22 is referred to as a proximal end side.

The operation unit 22 is a part that is joined to the proximal end side of the insertion unit 21 and that receives various operations from a doctor, or the like. As illustrated in FIG. 1, the operation unit 22 includes a curve knob 221 for an operation of causing the curve part 212 to curve and a plurality of operation members 222 for performing various operations. In the operation unit 22, a treatment tool insertion port 223 that communicates with the treatment tool insertion path and that is for inserting the treatment tool into the treatment tool insertion path is formed.

The universal cord 23 is a cable that extends from the operation unit 22 and in which a plurality of signal cables that transmit various signals, an optical fiber that transmits illumination light supplied from the light source device 6, etc., are arranged.

The connector 24 is provided at a distal end of the universal cord 23. The connector 24 and includes first to third connector parts 241 to 243 to which an ultrasound cable 3a, a video cable 4a, and an optical fiber cable 6a are connected, respectively.

The ultrasound imaging device 3 is electrically connected to the ultrasound endoscope 2 via the ultrasound cable 3a (refer to FIG. 1) and outputs the transmission signal that is a pulse signal to the ultrasound endoscope 2 via the ultrasound cable 3a and the reception signal that is an echo signal is input to the ultrasound imaging device 3 from the ultrasound endoscope 2. The ultrasound imaging device 3 generates an ultrasound image by performing given processing on the echo signal.

The endoscope imaging device 4 is electrically connected to the ultrasound endoscope 2 via the video cable 4a (refer to FIG. 1) and an image signal from the ultrasound endoscope 2 is input to the endoscope imaging device 4 via the video cable 4a. The endoscope imaging device 4 generates an endoscopic image by performing given processing on the image signal.

The display 5 is configured using liquid crystals or electro luminescence (EL), a projector, a cathode ray tube (CRT), or the like, and displays the ultrasound image that is generated by the ultrasound imaging device 3 or the endoscopic image that is generated by the endoscope imaging device 4.

The light source device 6 is connected to the ultrasound endoscope 2 via the optical fiber cable 6a (refer to FIG. 1) and supplies the illumination light that illuminates the inside of the subject to the ultrasound endoscope 2 via the optical fiber cable 6a.

The ultrasound transducer 7 is a radial transducer including, for example, 256 piezoelectric elements that are arranged along the circumference of the ultrasound transducer 7; however, the ultrasound transducer 7 may be a convex transducer or a linear transduce, and the number of piezoelectric elements is not limited. The ultrasound transducer 7 may include transducers that are arranged two-dimensionally. The ultrasound endoscope 2 is an endoscope in which a plurality of piezoelectric elements are arranged in a form of an array as the ultrasound transducer 7 and that performs electric scanning by electronically switching piezoelectric elements involved in transmission and reception and delaying transmission and reception of each of the piezoelectric elements.

### Configuration of Ultrasound Imaging device

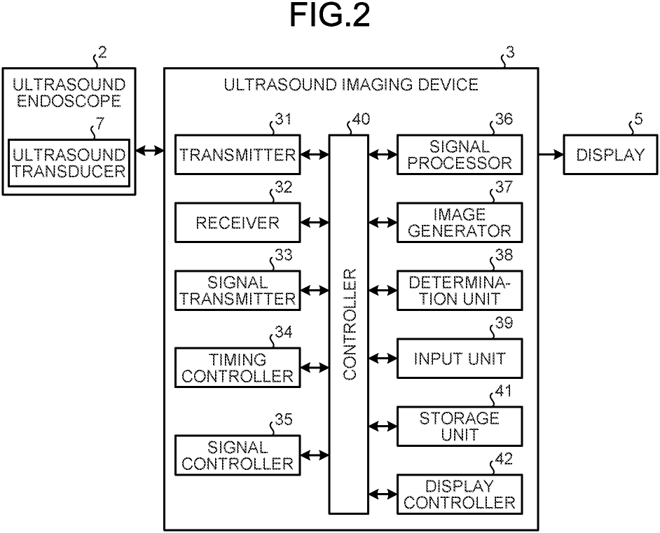

FIG. 2 is a block diagram illustrating a configuration of an ultrasound imaging device illustrated in FIG. 1. As illustrated in FIG. 2, the ultrasound imaging device 3 includes a transmitter 31, a receiver 32, a signal transmitter 33, a timing controller 34, a signal controller 35, a signal processor 36, an image generator 37, a determination unit 38, an input unit 39, a controller 40, a storage unit 41, and a display controller 42.

The transmitter 31 transmits transmission signals to the piezoelectric elements. Specifically, the transmitter 31 includes a high-voltage pulse generator, is electrically connected to the ultrasound endoscope 2, and transmits a transmission signal that is a high-voltage pulse that is generated by the high-voltage pulse generator according to a given waveform and given transmitting timing to each piezoelectric element of the ultrasound transducer 7. The transmitter 31 includes 256 transmitting circuits that transmit transmission signals to the piezoelectric elements and the transmitting circuits are connected to the piezoelectric elements, respectively. The frequency band of the pulse signals that the transmitter 31 transmit may be a wide band that almost covers a liner response frequency band of electric acoustic transduction from pulse signals into ultrasound pulses in the ultrasound transducer 7. The receiver 32 transmits various control signals that are output by the controller 40 to the ultrasound endoscope 2.

The receiver 32 receives echo signals from the piezoelectric elements. Specifically, the receiver 32 receives a transmission signal that is an electric echo signal from each of the piezoelectric elements of the ultrasound transducer 7 and generates and outputs data of a digital radio frequency (RF) signal (RF data below). The receiver 32 includes 256 receiving circuits that receive reception signals from the piezoelectric elements and the receiving circuits are connected to the piezoelectric elements, respectively. In other words, the number of piezoelectric elements that the ultrasound transducer 7 includes, the number of transmitting circuits that the transmitter 31 includes, and the number of receiving circuits that the receiver 32 includes are equal. When the transmitting timing and the receiving timing differ, the functions of the transmitting circuit and the receiving circuit may be realized by a single circuit and this circuit is referred to as a transceiver circuit below. The receiver 32 also has a function of receiving various types of information including an identifying ID from the ultrasound endoscope 2 and transmitting the various types of information to the controller 40.

The signal transmitter 33 includes a transmitting circuit that transmits a given signal to the piezoelectric element. The given signal is a high-voltage repolarization signal that repolarizes the piezoelectric element and is, for example, a unipolar pulse. Specifically, the signal transmitter 33 transmits a control signal to the high-voltage pulse generator of the transmitter 31, thereby transmitting the repolarization signal to the piezoelectric element via the transceiver circuit. Note that the signal transmitter 33 may include the high-voltage pulse generator. The given signal only needs to be a high-voltage signal that has an effect of repolarizing the piezoelectric element, and the given signal may be a bipolar pulse.

The timing controller 34 controls receiving timing at which the transmitter transmits the transmission signal and receiving timing at which the receiver receives the reception signal. The transmitting timing and the receiving timing are different sets of timing. The timing controller 34 is realized by using a central processing unit (CPU), various computing circuits, or the like.

The signal controller 35 causes the signal transmitter 33 to transmit the given signal to an area to which the transmitter 31 does not transmit the transmission signal at the transmitting timing or causes the signal transmitter 33 to transmit the given signal to transmit the given signal to an area from which the receiver 32 does not receive reception signals at the receiving timing. The signal controller 35 is realized using a CPU, various computing circuits, or the like.

The signal processor 36 generates digital B-mode reception data based on the RF data that is received from the receiver 32. Specifically, the signal processor 36 performs known processing, such as bandpass filter, envelope demodulation or logarithmic transformation, on the RF data, thereby generating the digital B-mode reception data. In logarithmic transformation, a common logarithm of a volume obtained by dividing the RF data by a reference voltage V_{c} is taken and expressed by a decibel value. The signal processor 36 outputs the generated B-mode reception data of one frame to the image generator 37. The signal processor 36 is realized using a CPU, various computing circuits, or the like.

The image generator 37 generates an ultrasound image (image data) based on the reception signal (RF data) that is received from the receiver 32. The image generator 37 performs signal processing using a known techniques, such as scan converter processing, gain processing, and contrast processing, on the B-mode reception data and performs data thinning corresponding to a data step width that is determined according to a range of display of an image on the display 5, thereby generating B-mode image data. In the scan converter processing, the direction of scanning the B-mode reception data from the direction of scanning ultrasound to a direction of display by the display 5. The B-mode image is a grayscale image in which the values of R (red), G (green) and B (blue) that are variables in the case where the RGB color system is employed coincide. The image generator 37 performs coordinate transformation in which rearrangement is performed to spatially express a scan area correctly on sets of B-mode reception data from the signal processor 36 and then performs interpolation processing between the sets of B-mode reception data, thereby filling the gap between the sets of B-mode reception data and generating the B-mode image data. The image generator 37 is realized using a central processing unit (CPU), various computing circuits, or the like.

The determination unit 38 determines whether the reception signal is a reflection signal from the subject. Specifically, when the voltage value of the reception signal exceeds a threshold, the determination unit 38 determines that the reception signal is a reflection signal from the subject. The determination unit 38 is realized using a central processing unit (CPU), various computing circuits, or the like.

The input unit 39 is realized using a user interface, such as a keyboard, a mouse, a touch panel or a track ball, and receives inputs of various types of information. The input unit 39 receives an input of an observation point, which is an input made by the user. The observation point is a position in an ultrasound image that the user wants to observe the most.

The controller 40 controls the entire ultrasound imaging system 1. The controller 40 is realized using a CPU having computing and controlling functions, various computing circuits, or the like. The controller 40 reads information that the storage unit 41 stores from the storage unit 41 and executes various types of computing processing relating to a method of operating the ultrasound imaging device 3, thereby controlling the ultrasound imaging device 3 in an integrated manner. It is also possible to configure the controller 40 using a common CPU, or the like, that is shared with the timing controller 34, the signal controller 35, the signal processor 36, the image generator 37, the determination unit 38, or the display controller 42.

The storage unit 41 stores various programs for causing the ultrasound imaging system 1 to operate and data containing various parameters necessary for operations of the ultrasound imaging system 1, etc. The storage unit 41 stores various programs containing an operation program for executing a method of operating the ultrasound imaging system 1. It is also possible to record the operation program in a computer-readable recording medium, such as a hard disk, a flash memory, a CD-ROM, a DVD-ROM, or a flexible disk, and distribute the operation program. It is also possible to acquire the above-described various programs by downloading the programs via a communication network. The communication network herein is realized by, for example, an existing public network, a local area network (LAN), a wide area network (WAN), or the like, and it does not matter whether the communication network is wired or wireless.

The storage unit 41 having the above-described configuration is realized using a read only memory (ROM) in which the various programs, etc., are installed in advance, a random access memory (RAM) that stores computing parameters of each process, data, etc., or the like.

The display controller 42 outputs data of an endoscopic image based on an imaging signal that is generated by the imaging device and data of an ultrasound image that is generated by the image generator 37 based on an electric reception signal that is generated by the ultrasound transducer 7 to the display 5 and causes the display 5 to make a display. Furthermore, the data of the endoscopic image and the data of the ultrasound image with various types of information being superimposed thereon are output to the display 5 and the display 5 is caused to make a display. The display controller 42 causes the display 5 to display an area (piezoelectric elements) to which the signal controller 35 causes the signal transmitter 33 to transmit the repolarization signal. The display controller 42 is realized using a CPU, various computing circuits, or the like.

### Operations of Ultrasound Imaging device

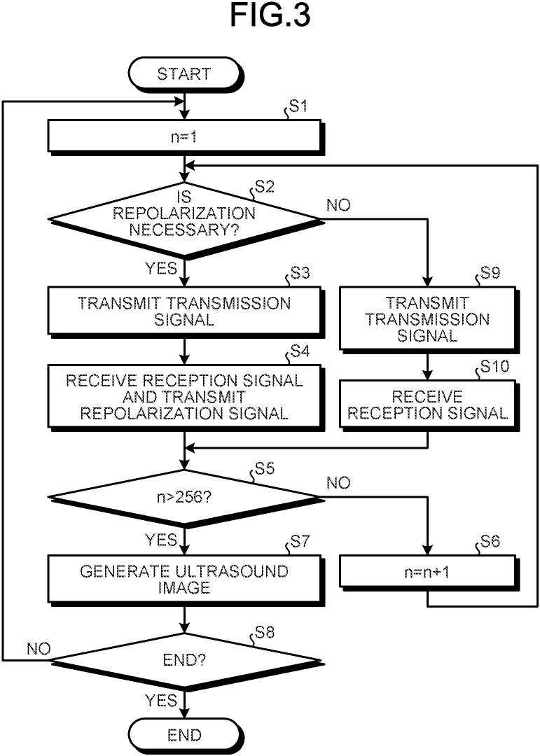

Operations of the ultrasound imaging device 3 will be described next. FIG. 3 is a flowchart illustrating an overview of a process that the ultrasound imaging device executes. As illustrated in FIG. 3, first of all, the controller 40 sets a variable n corresponding to a piezoelectric element number at n=1 (step S1).

The determination unit 38 then determines whether repolarization is necessary with respect to each piezoelectric element of the ultrasound transducer 7 (step S2). Specifically, the determination unit 38 determines whether repolarization is necessary with respect to each of the 256 piezoelectric elements of the ultrasound transducer 7 based on a determination reference on whether the voltage of the reception signal that is received by the receiver 32 previously exceeds a threshold and whether a given time elapses after transmission of the repolarization signal.

At the start of observation, when the receiver 32 has not received the reception signal or when it is impossible to determine a time after transmission of the repolarization signal, the determination unit 38 may determine to transmit the repolarization signal to all the piezoelectric elements.

When the determination unit 38 determines that repolarization is necessary (YES at step S2), the timing controller 34 performs control such that the transmitting timing and the receiving timing are different sets of timing (the transmitting timing and the receiving timing do not overlap) and transmits and receives ultrasound.

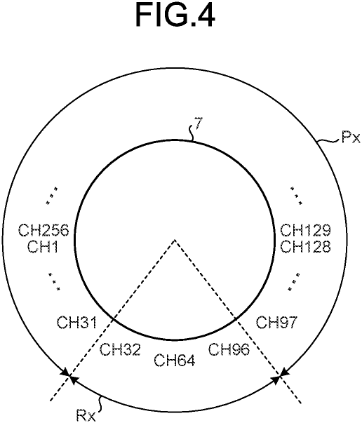

First of all, at the transmitting timing, the transmitter 31 transmits the transmission signal to the piezoelectric element corresponding to the variable n (step S3). FIG. 4 is a diagram for describing a relationship of connection between the piezoelectric elements and the transceiver circuits. As illustrated in FIG. 4, the 256 piezoelectric elements of the ultrasound transducer 7 are arrayed along the circumference and the piezoelectric elements are connected to 256 transceiver circuits CH1 to CH256, respectively. The transmitter 31 transmits the transmission signal to a piezoelectric element that is connected to a transceiver circuit CHn. FIG. 4 illustrates an example in which n=64 and the transmitter 31 transmits the transmission signal to the piezoelectric element that is connected to the transceiver circuit CH64. Note that the transmitter 31 may transmit the transmission signals to a plurality of piezoelectric elements around a piezoelectric element that is connected to CHn.

Thereafter, at the receiving timing, the receiver 32 receives a reception signal from the piezoelectric element and the signal controller 35 causes the signal transmitter 33 to transmit the repolarization signal to the piezoelectric element that is positioned in an area from which the receiver 32 does not receive reception signals (step S4). In the example illustrated in FIG. 4, 32 transceiver circuits adjacent to and anterior to CH64 to which the transmitter 31 transmit the transmission signals and 32 transceiver circuits adjacent to and posterior to CH64 receive reception signals. The reception signals from the 65 piezoelectric elements in total are summed to generate a reception signal corresponding to the single piezoelectric element that is connected to the transceiver circuit CH64. In other words, the transceiver circuits CH32 to CH96 are receiving channels (Rx) that are used to receive reception signals. At the receiving timing, the transceiver circuit CH1 to CH31 and the transceiver circuits CH97 to CH256 are not used to generate an ultrasound image. The signal controller 35 thus causes the signal transmitter 33 to transmit the repolarization signals to the repolarization channels (Px) CH1 to CH31 and CH97 to CH256 corresponding to the area from which the receiver 32 does not receive reception signals at the receiving timing.

The controller 40 determines whether the variable n>256 (step S5). When the controller 40 determines that n>256 is not satisfied (NO at step S5), the controller 40 determines n=n+1 and returns to step S2.

On the other hand, when the controller 40 determines the variable n>256 (YES at step S5), the image generator 37 generates an ultrasound image based on the reception signal that is received by the receiver 32 (step S7).

Thereafter, the controller 40 determines whether to end observation by the ultrasound imaging device 3 (step S8) .

When the controller 40 determines to end observation by the ultrasound imaging device 3 (YES at step S8), the controller 40 ends the sequential process.

On the other hand, when the controller 40 determines not to end observation by the ultrasound imaging device 3 (NO at step S8), the controller 40 returns to step S1 and continues the process.

At step S2, when the determination unit 38 determines that repolarization is unnecessary (NO at step S2), at the transmitting timing, the transmitter 31 transmits the transmission signal to the piezoelectric element corresponding to the variable n (step S9). Furthermore, at the receiving timing, the receiver 32 receives reception signals from piezoelectric elements (step S10). At the receiving timing, the signal controller 35 prevents the signal transmitter 33 from transmitting the repolarization signal.

According to the first embodiment described above, transmitting the repolarization signals to the piezoelectric elements from which the receiver 32 does not receive reception signals at the receiving timing makes it possible to recover acoustic characteristics while generating an ultrasound image. The ultrasound imaging device 3 transmits the repolarization signal for N times while the image generator 37 generates a single ultrasound image, thereby recovering acoustic characteristics. In other words, the signal controller 35 causes the signal transmitter 33 to transmit the repolarization signals when the image generator 37 is generating an ultrasound image. As a result, acoustic characteristics are prevented from deteriorating when the ultrasound imaging device 3 is used (when observation is performed).

According to the first embodiment described above, the signal controller 35 transmits the repolarization signal to a piezoelectric element from which the receiver 32 does not receive a reception signal at the receiving timing; however, the signal controller 35 may transmit the repolarization signal to a piezoelectric element to which the transmitter 31 does not transmit the transmission signal at the transmitting timing. In this case, in order to prevent the repolarization signal from having an effect on the ultrasound image, it is preferable that the repolarization signals be transmitted to the transceiver circuits away from the piezoelectric element to which the transmitter 31 transmits the transmission signal (for example, in the situation according to FIG. 4, the transceiver circuits CH129 to CH256 that are positioned on a side opposite to CH64 to which the transmission signal is transmitted).

The signal controller 35 causes the signal transmitter 33 to transmit the repolarization signals to all the piezoelectric elements corresponding to the area from which the receiver 32 does not receive reception signals at the receiving timing; however, the transmission is not limited thereto. The signal controller 35 may cause the signal transmitter 33 to transmit the repolarization signal to part of the piezoelectric elements from which the receiver 32 does not receive reception signals at the receiving timing. For example, in the situation illustrated in FIG. 4, the signal controller 35 may cause the signal transmitter 33 to transmit the repolarization signals to part of the transceiver circuits (for example, the transceiver circuits H12 to CH256) corresponding to the area from which the receiver 32 does not receive transmission signals at the receiving timing. In this case, because there are piezoelectric elements that are not used for any of transmission and reception and repolarization, it is possible to prevent an increase in the surface temperature of the ultrasound transducer 7 due to continuous use of the piezoelectric elements, a decrease in the acoustic output due to simultaneous use of a large number of piezoelectric elements, etc.

The signal controller 35 may cause the signal transmitter 33 to transmit the repolarization signal at given periods. In this case, setting a period during which the repolarization signal is not transmitted makes it possible to prevent an increase in the surface temperature of the ultrasound transducer 7 due to continuous use of the piezoelectric elements, a reduction in the acoustic output due to simultaneous use of a large number of piezoelectric elements, etc.

The determination unit 38 may determine whether the reception signal is a reflection signal from the subject. Specifically, the determination unit 38 determines that the reception signal is not a reflection signal from the subject when the voltage value of the reception signal exceeds a threshold. This is because, when the ultrasound transducer 7 is not making contact with the subject and there is an air layer between the ultrasound transducer 7 and the subject, the transmission signal reflects off a lens reflection surface of the acoustic lens of the ultrasound transducer 7 and the voltage value of the reception signal increases. The signal controller 35 then causes the signal transmitter 33 to transmit the repolarization signal to the piezoelectric element on which the determination unit 38 determines that the reception signal is not a reflection signal from the subject. As a result, because it is possible to transmit the repolarization signal to the piezoelectric element that is unable to generate an ultrasound image correctly because the ultrasound transducer 7 does not make contact with the subject, it is possible to receiver acoustic characteristics without an effect on the ultrasound image. Note that, because the piezoelectric element to which the repolarization signal is transmitted is unable to generate a correct ultrasound image, the display controller 42 may cause the display 5 to display the piezoelectric element to which the repolarization signal is transmitted.

### Modification

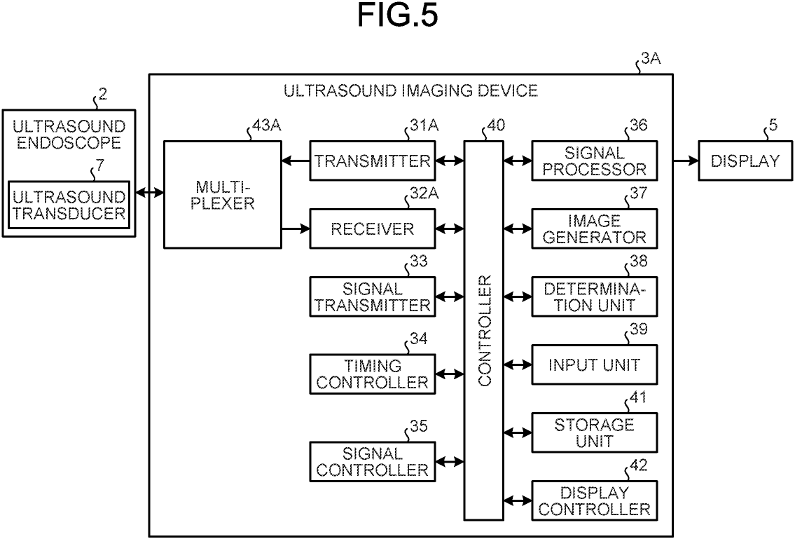

FIG. 5 is a block diagram illustrating a configuration of an ultrasound imaging device according to a modification. As illustrated in FIG. 5, an ultrasound imaging device 3A includes a transmitter 31A including 128 transmitting circuits that transmit transmission signals to piezoelectric elements, a receiver 32A including 128 receiving circuits that receive reception signals from the piezoelectric elements, and a multiplexer 43A that is a switch that switches connection among the transmitting circuits, the receiving circuits, and the piezoelectric elements. An example in which the ultrasound imaging device 3A includes 128 transceiver circuits configured by integrating the transmitting circuits and the receiving circuits will be described.

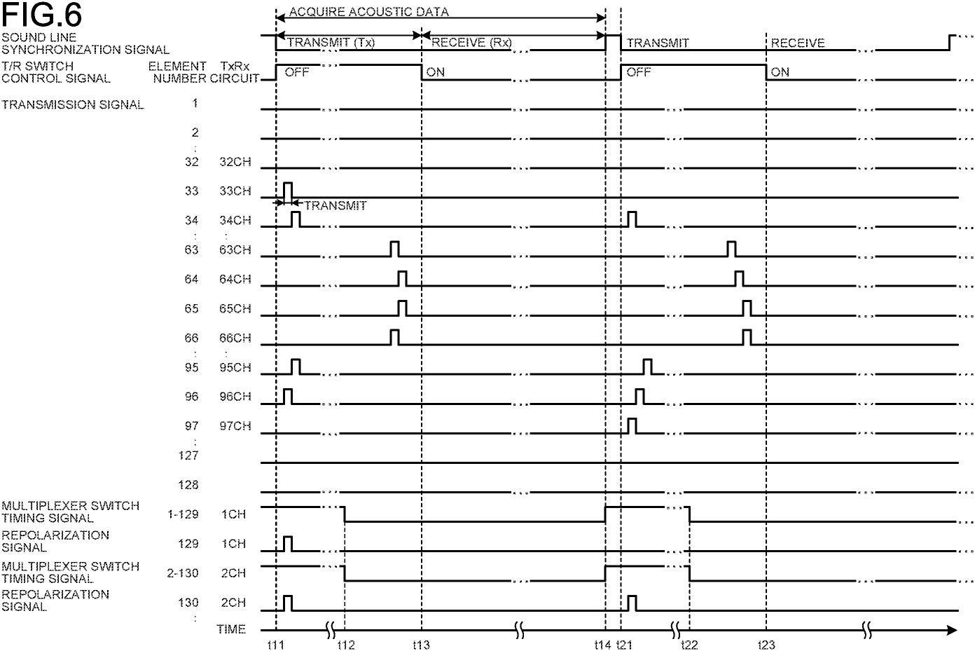

FIG. 6 is a timing chart illustrating timing at which each signal is transmitted or received. FIG. 6 presents the signal type, the element number (the number of piezoelectric element), the number of TxRx circuit (transceiver circuit) from the left. First of all, the timing controller 34 transmits a sound ray synchronization signal at given periods and accordingly the sets of timing at which a signal corresponding to one sound ray (one piezoelectric element) is transmitted and received synchronize. Furthermore, the timing controller 34 switches transmitting timing (Tx) at which a T/R switch control signal corresponds to OFF and receiving timing (Rx) at which the T/R switch control signal corresponds to ON. In other words, the transmitting timing (Tx) and the receiving timing (Rx) are different sets of timing (do not overlap).

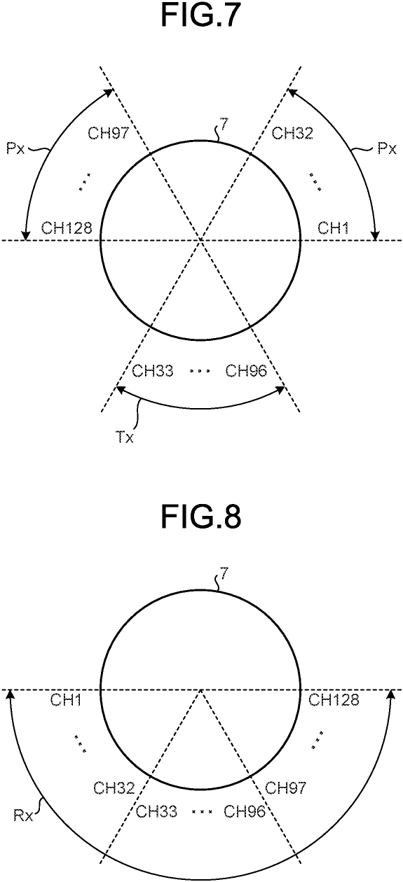

At times t11 to t13, the transmitter 31 transmits transmission signals to piezoelectric elements with element numbers EL33 to EL96 via transceiver circuits CH33 to CH96. FIG. 7 is a diagram for describing a relationship of connection of the piezoelectric elements and the transceiver circuits at transmitting timing. As illustrated in FIG. 7, the transceiver circuits CH33 to CH96 are transmitting channels (Tx) that are used to transmit the transmission signal. Transceiver circuits CH1 to CH32 and transceiver circuits CH97 to CH128 are not used to transmit the transmission signal. Thus, the timing controller 34 controls the multiplexer 43A by transmitting a multiplexer switch timing signal, thereby switching the piezoelectric elements that are connected to the transceiver circuits CH1 to CH32 from the piezoelectric elements with element numbers EL1 to EL32 to piezoelectric elements with element numbers EL129 to EL160. Furthermore, the signal controller 35 causes the signal transmitter 33 to transmit repolarization signals to the piezoelectric elements EL129 to EL160 via the transceiver circuits CH1 to CH32. Similarly, the timing controller 34 controls the multiplexer 43A by transmitting a multiplexer switch timing signal, thereby switching the piezoelectric elements that are connected to the transceiver circuits CH97 to CH128 from the piezoelectric elements with element numbers EL97 to EL128 to piezoelectric elements with element numbers EL225 to EL256. Furthermore, the signal controller 35 causes the signal transmitter 33 to transmit the repolarization signals to the piezoelectric elements with element numbers EL225 to EL256 via the transceiver circuits CH97 to CH 128. As a result, the transceiver circuits CH1 to CH32 that are connected to the piezoelectric elements with element numbers EL129 to EL160 and transceiver circuits CH97 to CH128 that are connected to the piezoelectric elements with element numbers EL225 to EL256 serve as repolarization channels (Px) via which the repolarization signals are transmitted.

Thereafter, at time t12, the piezoelectric elements that are connected to the transceiver circuits CH1 to CH32 and the transceiver circuits CH97 to CH128 return to the piezoelectric elements with element numbers EL1 to EL32 and the piezoelectric elements with element numbers EL97 to EL128. During times t13 to t14, the receiver 32 receives reception signals from the piezoelectric elements with element numbers EL1 to EL128 via the transceiver circuits CH1 to CH128. FIG. 8 is a diagram for describing a relationship of connection of the piezoelectric elements and the transceiver circuits at receiving timing. As illustrated in FIG. 8, the transceiver circuits CH1 to CH128 are receiving channels (Rx) that are used to receive reception signals.

Moving to the next sound line, during times t21 to t23, the transmitter 31 transmits the transmission signals to the piezoelectric elements with element numbers EL34 to EL97 via the transceiver circuits CH34 to CH97. The signal controller 35 causes the signal transmitter 33 to transmit the repolarization signals to the piezoelectric elements with element numbers EL130 to EL161 and the piezoelectric elements with element numbers EL226 to EL1 via the transceiver circuits CH2 to CH33 and the transceiver circuits CH98 to CH1. During the times t23 to t24, the receiver 32 receives reception signals from the piezoelectric elements with element numbers EL2 to EL129 via the transceiver circuits CH2 to CH1.

According to the modification described above, even when the relationship of connection between the piezoelectric elements and the transceiver circuits is switched by the multiplexer 43A, transmitting the repolarization signals to the piezoelectric elements to which the transmitter 31 does not transmit the transmission signals at the transmitting timing makes it possible to recover the acoustic characteristics while generating an ultrasound image.

### Second Embodiment

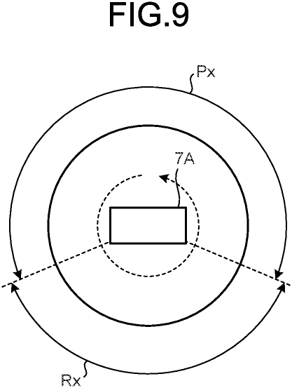

FIG. 9 is a diagram for describing a positional relationship of a piezoelectric element. As illustrated in FIG. 9, an ultrasound transducer 7A of an ultrasound imaging device according to a second embodiment includes a piezoelectric element and the piezoelectric element is mechanically scanned. The ultrasound transducer 7A is a radial transducer that rotates the piezoelectric element.

The determination unit 38 determines whether a reception signal is a reflection signal from a subject. Specifically, when the voltage value of the reception signal exceeds a threshold, the determination unit 38 determines that the reception signal is not a reflection signal from the subject. This is because, when the ultrasound transducer 7 is not making contact with the subject and there is an air layer between the ultrasound transducer 7 and the subject, the transmission signal reflects off a lens reflection surface of the acoustic lens of the ultrasound transducer 7 and the voltage value of the reception signal increases.

In the example illustrated in FIG. 9, an area on which the determination unit 38 determines that the reception signal is a reflection signal from the subject serves as receiving channels (Rx) that are used to receive reception signals. On the other hand, an area on which the determination unit 38 determines that the reception signal is not a reflection signal from the subject serves as repolarization channels (Px) via which the repolarization signal is transmitted. As a result, because it is possible to transmit the repolarization signal to the piezoelectric element that is unable to generate an ultrasound image correctly because the ultrasound transducer 7 is not making contact with the subject, it is possible to recover acoustic characteristics without an effect on an ultrasound image. The piezoelectric element to which the repolarization signal is transmitted is unable to generate a correct ultrasound image, and therefore the display controller 42 may cause the display 5 to display an area to which the repolarization signal is transmitted. The display controller 42 may cause the display 5 to display the area to which the repolarization signal is transmitted in a changed color or may cause the display 5 to display the area with a shade superimposed thereon.

The signal controller 35 causes the signal transmitter 33 to transmit the repolarization signals to all the piezoelectric elements corresponding to the area from which the receiver 32 does not receive reception signals at the receiving timing; however, the transmission is not limited thereto. The signal controller 35 may select an area to which the signal transmitter 33 is caused to transmit the repolarization signal and an area to which the signal transmitter 33 is prevented from transmitting the repolarization signal. For example, in the situation illustrated in FIG. 9, the signal controller 35 may cause the signal transmitter 33 to transmit the repolarization signal to part of an area (for example, an area away from Rx by a given amount or larger) corresponding to the area from which the receiver 32 does not receive transmission signals at the receiving timing. In this case, there are piezoelectric elements that are not used for any of transmission and reception and repolarization. As a result, because each piezoelectric element has a period during which the piezoelectric element is not used for any of transmission and reception and repolarization, it is possible to prevent an increase in the surface temperature of the ultrasound transducer 7 due to continuous use of the piezoelectric elements, a decrease in the acoustic output, etc.

The signal controller 35 may cause the signal transmitter 33 to transmit the repolarization signal at given periods. In this case, setting a period during which the repolarization signal is not transmitted in each piezoelectric element makes it possible to prevent an increase in the surface temperature of the ultrasound transducer 7 due to continuous use of the piezoelectric elements, a reduction in the acoustic output, etc.

In the above-described embodiment, the example in which the repolarization signal is transmitted to the piezoelectric elements in the state where ultrasound is transmitted and received via piezoelectric elements and an ultrasound image that is a B-mode image is being generated has been described; however, the transmission is not limited thereto. For example, the repolarization signal may be transmitted to the piezoelectric elements in the state where an ultrasound image is being generated by THI (Tissue Harmonic Imaging) that generates an ultrasound image using harmonic waves. When HIFU (High Intensity Focused Ultrasound) in which a subject tissue is cauterized by ultrasound that is applied from piezoelectric elements is performed, the signal controller 35 may transmit the repolarization signal to an area to which the transmitter 31 does not transmit a drive signal for performing HIFU. Similarly, when elastography in which stiffness of living tissue is measured is performed, the signal controller 35 may transmit the repolarization signal to an area to which the transmitter 31 does not transmit a signal for applying a push pulse.

According to the disclosure, it is possible to realize an ultrasound imaging device that makes it possible to recover acoustic characteristics also when the ultrasound imaging device is used, an ultrasound imaging system, and a method of operating an ultrasound imaging device.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound imaging device comprising:
a first transmitter configured to transmit a transmission signal to at least one piezoelectric element;
a receiver configured to receive a reception signal from the at least one piezoelectric element;
a second transmitter configured to transmit a given signal to the at least one piezoelectric element;
a timing controller configured to control a transmitting timing at which the first transmitter transmits the transmission signal and a receiving timing at which the receiver receives the reception signal; and
a signal controller configured to cause the second transmitter to transmit the given signal to a first area to which the first transmitter does not transmit the transmission signal at the transmitting timing or cause the second transmitter to transmit the given signal to a second area from which the receiver does not receive the reception signal at the receiving timing.

2. The ultrasound imaging device according to claim 1, wherein the given signal is a repolarization signal that repolarizes the at least one piezoelectric element.

3. The ultrasound imaging device according to claim 1, wherein the given signal is a unipolar pulse.

4. The ultrasound imaging device according to claim 1, wherein the transmitting timing and the receiving timing are different sets of timing.

5. The ultrasound imaging device according to claim 1, wherein the signal controller is configured to cause the second transmitter to transmit the given signal to part of the first area or cause the second transmitter to transmit the given signal to part of the second area .

6. The ultrasound imaging device according to claim 1, wherein the signal controller is configured to cause the second transmitter to transmit the given signal at given periods.

7. The ultrasound imaging device according to claim 1, further comprising an image generator configured to generate an ultrasound image based on the reception signal,
wherein the signal controller is configured to cause the second transmitter to transmit the given signal when the image generator is generating the ultrasound image.

8. The ultrasound imaging device according to claim 1, further comprising a display controller configured to cause a display to display a third area to which the signal controller causes the second transmitter to transmit the given signal.

9. The ultrasound imaging device according to claim 1, further comprising a determination circuit configured to determine whether the reception signal is a reflection signal from a subject,
wherein the signal controller is configured to cause the second transmitter to transmit the given signal to a fourth area on which the determination circuit determines that the reception signal is not the reflection signal from the subject.

10. The ultrasound imaging device according to claim 9, wherein, the determination circuit is configured to, when a voltage value of the reception signal exceeds a threshold, determine that the reception signal is not the reflection signal from the subject.

11. The ultrasound imaging device according to claim 1, wherein the at least one piezoelectric element comprises a plurality of piezoelectric elements, and
the signal controller is configured to cause the second transmitter to transmit the given signal to a piezoelectric element to which the first transmitter does not transmit the transmission signal among the plurality of piezoelectric elements at the transmitting timing or cause the second transmitter to transmit the given signal to a piezoelectric element from which the receiver does not receive the reception signal among the plurality of piezoelectric elements at the receiving timing.

12. The ultrasound imaging device according to claim 11, wherein the first transmitter includes a plurality of transmitting circuits each configured to transmit the transmission signal to one of the plurality of piezoelectric elements, and
the number of the plurality of piezoelectric elements and the number of the plurality of transmitting circuits are equal.

13. The ultrasound imaging device according to claim 11, wherein the first transmitter includes
a plurality of transmitting circuits each configured to transmit the transmission signal to one of the plurality of piezoelectric elements, and
the ultrasound imaging device further comprises a switch configured to switch connection between the plurality of transmitting circuit and the plurality of piezoelectric element.

14. The ultrasound imaging device according to claim 1, wherein the at least one piezoelectric element comprises a plurality of piezoelectric elements, and
the plurality of piezoelectric elements are arrayed along a circumference.

15. A method of operating an ultrasound imaging device including
a first transmitter configured to transmit a transmission signal to at least one piezoelectric element;
a receiver configured to receive a reception signal from the at least one piezoelectric element; and
a second transmitter configured to transmit a given signal that repolarizes the piezoelectric element to the at least one piezoelectric element, the method comprising:
by a timing controller, controlling a transmitting timing at which the first transmitter transmits the transmission signal and a receiving timing at which the receiver receives the reception signal; and
by a signal controller, causing the second transmitter to transmit the given signal to a first area to which the first transmitter does not transmit the transmission signal at the transmitting timing or causing the second transmitter to transmit the given signal to a second area from which the receiver does not receive the reception signal at the receiving timing.

16. A non-transitory computer-readable recording medium with an executable program stored thereon, the program causing an ultrasound imaging device to execute:
causing a first transmitter to transmit a transmission signal to at least one piezoelectric element;
causing a receiver to receive a reception signal from the at least one piezoelectric element;
causing a second transmitter to transmit a given signal to the at least one piezoelectric element;
causing a timing controller to control a transmitting timing at which the first transmitter transmits the transmission signal and a receiving timing at which the receiver receives the reception signal; and
causing a signal controller to cause the second transmitter to transmit the given signal to a first area to which the first transmitter does not transmit the transmission signal at the transmitting timing or cause the second transmitter to transmit the given signal to a second area from which the receiver does not receive the reception signal at the receiving timing.

17. An ultrasound imaging system comprising:
an ultrasound probe including at least two ultrasound transducers; and
a processor configured to
cause the at least two ultrasound transducers included in a first area to transmit and receive an ultrasound at a first timing, and
transmit a repolarization signal to the at least two ultrasound transducers included in a second area at the first timing.
